SEQUENCE LISTING (25 sequences)

SEQ ID NO: 1 - DNA, 156 bp, BK polyomavirus
tcaggagagtttatagaaaaaactattgccccaggaggtgctaatcaaagaactgctcctcaatggatgttgcctttacttctaggcctgtacgggactgtaacacctgctcttgaagcatatgaagatggccccaaccaaaagaaaaggagagtg

SEQ ID NO: 2 - DNA, 22 bp, Artificial Sequence
  Description of Artificial Sequence Synthetic primer
cccaggaggtgctaatcaaaga

SEQ ID NO: 3 - DNA, 23 bp, Artificial Sequence
  Description of Artificial Sequence Synthetic primer
tcatatgcttcaagagcaggtgt

SEQ ID NO: 4 - DNA, 21 bp, Artificial Sequence
  Description of Artificial Sequence Synthetic oligonucleotide
gctcctcaatggatgttgcct

SEQ ID NO: 5 - DNA, 24 bp, Artificial Sequence
  Description of Artificial Sequence Synthetic probe
cttctaggcctgtacgggactgta

SEQ ID NO: 6 - DNA, 23 bp, Artificial Sequence
  Description of Artificial Sequence Synthetic primer
tcanatgcttcaagagcaggtgt

SEQ ID NO: 7 - DNA, 23 bp, Artificial Sequence
  Description of Artificial Sequence Synthetic primer
aaaaactattgccccaggaggtg

SEQ ID NO: 8 - DNA, 23 bp, Artificial Sequence
  Description of Artificial Sequence Synthetic primer
ccccaggaggtgctaatcaaaga

SEQ ID NO: 9 - DNA, 23 bp, Artificial Sequence
  Description of Artificial Sequence Synthetic oligonucleotide
tacagtcccgtacaggcctagaa

SEQ ID NO: 10 - DNA, 25 bp, Artificial Sequence
  Description of Artificial Sequence Synthetic probe
aaggcaacatccattgaggagcagt

SEQ ID NO: 11 - DNA, 17 bp, Artificial Sequence
  Description of Artificial Sequence Synthetic probe
aggcaacatccattgag

SEQ ID NO: 12 - DNA, 24 bp, Artificial Sequence
  Description of Artificial Sequence Synthetic primer
tcaggagagtttatagaaaaaact

SEQ ID NO: 13 - DNA, 23 bp, Artificial Sequence
  Description of Artificial Sequence Synthetic probe
tacagtcccgtacaaccctaaaa

SEQ ID NO: 14 - DNA, 16 bp, Artificial Sequence
  Description of Artificial Sequence Synthetic probe
ccgtacaggcctagaa

SEQ ID NO: 15 - DNA, 17 bp, Artificial Sequence
  Description of Artificial Sequence Synthetic probe
cgtacaaccctaaaagt

SEQ ID NO: 16 - DNA, 23 bp, Artificial Sequence
  Description of Artificial Sequence Synthetic probe
acagtcccgtacaggcctagaag

SEQ ID NO: 17 - DNA, 19 bp, Artificial Sequence
  Description of Artificial Sequence Synthetic primer
tttggctttttgggagctg

SEQ ID NO: 18 - DNA, 23 bp, Artificial Sequence
  Description of Artificial Sequence Synthetic primer
ccctggacactctccttttcttt

SEQ ID NO: 19 - DNA, 21 bp, Artificial Sequence
  Description of Artificial Sequence Synthetic primer
ccccaggaggtgctaatcaaa

SEQ ID NO: 20 - DNA, 24 bp, Artificial Sequence
  Description of Artificial Sequence Synthetic primer
atgcttcaagagcaggtgttacag

SEQ ID NO: 21 - DNA, 23 bp, Artificial Sequence
  Description of Artificial Sequence Synthetic probe
ctgctcctcaatggatgttgcct

SEQ ID NO: 22 - DNA, 22 bp, Artificial Sequence
  Description of Artificial Sequence Synthetic primer
cccaggaggtgcnaatcaaaga

SEQ ID NO: 23 - DNA, 24 bp, Artificial Sequence
  Description of Artificial Sequence Synthetic probe
cttttagggttgtacgggactgta

SEQ ID NO: 24 - DNA, 18 bp, Artificial Sequence
  Description of Artificial Sequence Synthetic primer
ctgcccctggacactctc

SEQ ID NO: 25 - DNA, 20 bp, Artificial Sequence
  Description of Artificial Sequence Synthetic primer
agctgcccctggacactctc

(19) United States
(12) Patent Application Publication
(10) Pub. No.: US 2011/0091866 A1
(43) Pub. Date: Apr. 21, 2011

(54) DETECTION OF POLYOMAVIRUS

(76) Inventor: Kosuke Ken Iwaki, Palos Verdes Estates, CA (US)

(21) Appl. No.: 12/918,055

(22) PCT Filed: Feb. 19, 2009
(86) PCT No.: PCT/US2009/001032

§ 371 Date: Nov. 5, 2010

Related U.S. Application Data
(60) Provisional application No. 61/064,166, filed on Feb. 20, 2008.

(51) Int. Cl. C12Q 1/70; C12Q 1/68; C07H 21/00

(52) U.S. Cl. 435/5; 435/6; 536/24.31

(57) ABSTRACT

Methods and kits are provided for testing for the presence or absence of a polyomavirus, such as BKV, in a sample. The methods and kits are useful for quantifying BKV and differentiating BKV from JCV.

### CROSS-REFERENCE TO RELATED PATENT APPLICATION

This application claims the benefit of the priority date of U.S. provisional patent application No. 61/064,166, filed Feb. 20, 2008, the disclosure of which is incorporated herein by reference in its entirety.

### BACKGROUND

Human polyomaviruses JC and BK are ubiquitous in the population. Primary infections with these viruses are usually asymptomatic and may result in transient viruria. Following primary infection, JC virus (JCV) and BK virus (BKV) both establish latency in renal tissues and in B lymphocytes (G. Lecatsas, B. D. Schoub, A. R. Rabson, and M. Joffe, Letter, Lancet 2:907-908, 1976). Polyomavirus-related disease is largely associated with immunological impairment, and rapid detection and differentiation of the etiological agent in immunocompromised patients are important to assist with clinical management. JCV is the causative agent of the neurological disease progressive multifocal leukoencephalopathy, which occurs primarily in AIDS patients, whereas BKV-associated disease includes hemorrhagic cystitis, ureteral stenosis, and other urinary tract disease, which are most commonly found in transplant patients undergoing immunosuppressive therapy.

Traditional methods for detecting and identifying polyomaviruses include serologic methods, virus isolation by cell culturing and electron microscopy. Recently, studies have shown PCR to be an effective tool for detecting polyomaviruses in a range of clinical samples.

A major obstacle, however, in developing an effective detection assay for polyomavirus has been the large number of intra-species polymorphisms in the nucleotide sequences of BKV and JCV. Nucleotide polymorphisms such as SNPs, insertions and deletions heretofore have precluded the development of a reliable means to detect infection. More robust assays, therefore, are needed.

### SUMMARY

According to one aspect of the invention, methods are provided for testing the presence or absence of a polyomavirus in a sample, comprising testing the sample for the presence or absence of a nucleic acid having the sequence of SEQ ID NO: 1, its reverse complement, or a sequence having 90% or more sequence homology with SEQ ID NO: 1.

In some embodiments, the method further includes amplifying the nucleic acid of SEQ ID NO: 1 or its reverse complement or a portion of either and then testing for the presence or absence of the resulting amplicon. In some aspects, the testing step includes contacting the sample with at least one oligonucleotide probe capable of hybridizing to the nucleic acid of SEQ ID NO: 1 or its reverse complement under stringent conditions, or by conducting a melting curve analysis.

In one embodiment, the methods comprise the use of at least amplification primers SEQ ID NO: 2 and SEQ ID NO: 3 and the testing step comprises the use of at least oligonucleotide probes SEQ ID NO: 4 and SEQ ID NO: 5.

In another embodiment, the methods comprise the use of at least amplification primers SEQ ID NO: 2 and SEQ ID NO: 6 and the testing step comprises the use of at least oligonucleotide probes SEQ ID NO: 4 and SEQ ID NO: 5.

In another embodiment, the methods comprise the use of at least amplification primers SEQ ID NO: 2 and SEQ ID NO: 3 and the testing step comprises the use of at least oligonucleotide probes SEQ ID NO: 4 and SEQ ID NO: 23;

In still another embodiment, the methods comprise the use of at least amplification primers SEQ ID NO: 2 and SEQ ID NO: 6 and the testing step comprises the use of at least oligonucleotide probes SEQ ID NO: 4 and SEQ ID NO: 23;

In yet another embodiment, the methods comprise the use of at least amplification primers SEQ ID NO: 8 and SEQ ID NO: 9. In one aspect, the testing step comprises the use of a cyanine dye that binds to double-stranded DNA.

In another embodiment, the methods comprise the use of at least amplification primers SEQ ID NO: 4 and SEQ ID NO: 6 and the testing step comprises the use of at least oligonucleotide probes SEQ ID NO: 9 and SEQ ID NO: 13. Probes SEQ ID NO: 9 and SEQ ID NO: 13 can be used individually or simultaneously in the testing step.

In another embodiment, the methods comprise the use of at least amplification primers SEQ ID NO: 4 and SEQ ID NO: 6 and the testing step comprises the use of at least oligonucleotide probes SEQ ID NO: 14 and SEQ ID NO: 15.

In another embodiment, the methods comprise the use of at least amplification primers BKV_{—}5.2 and BKV_{—}5.1. These primers are located near the tail of the VP2/3 gene. Although VP2/3 and VP1 have separate open reading frames (ORF), BKV 5.2 and BKV 5.1 primers amplify a region of the VP2/3 gene that overlaps with the beginning of the VP1 gene.

In another aspect, kits are provided that comprise at least one oligonucleotide probe capable of hybridizing to the nucleic acid of SEQ ID NO: 1 under stringent conditions. In one aspect, the kit further comprises amplification primers for amplifying the nucleic acid of SEQ ID NO: 1, a complement or transcript or a portion thereof.

In one embodiment, the kit comprises amplification primers SEQ ID NO: 2 and SEQ ID NO: 3 and oligonucleotide probes SEQ ID NO: 4 and SEQ ID NO:5.

In another embodiment, the kit comprises amplification primers SEQ ID NO: 2 and SEQ ID NO: 6 and oligonucleotide probes SEQ ID NO: 4 and SEQ ID NO: 5.

In another embodiment, the kit comprises amplification primers SEQ ID NO: 2 and SEQ ID NO: 3 and oligonucleotide probes SEQ ID NO: 4 and SEQ ID NO: 23;

In still another embodiment, the kit comprises amplification primers SEQ ID NO: 2 and SEQ ID NO: 6 and oligonucleotide probes SEQ ID NO: 4 and SEQ ID NO: 23;

In another embodiment, the kit comprises amplification primers SEQ ID NO: 4 and SEQ ID NO: 6 and oligonucleotide probes SEQ ID NO: 9 and SEQ ID NO: 13. Probes SEQ ID NO: 9 and SEQ ID NO: 13 can be used individually or simultaneously.

In one embodiment, the kit comprises amplification primers SEQ ID NO: 4 and SEQ ID NO: 6 and oligonucleotide probes SEQ ID NO: 14 and SEQ ID NO: 15.

In one embodiment, the kit comprises amplification primers SEQ ID NO: 8 and SEQ ID NO: 9.

In another embodiment, the kit comprises amplification primers BKV_{—}5.2 and BKV_{—}5.1. These primers are located near the tail of the VP2/3 gene. Although VP2/3 and VP1 have separate open reading frames (ORF), BKV 5.2 and BKV 5.1 primers amplify a region of the VP2/3 gene that overlaps with the beginning of the VP1 gene.

In some embodiments, at least one of the amplification primers specifically binds to the BKV genomic DNA under stringent conditions. In one embodiment, at least one of the oligonucleotide probes specifically binds to the BKV genomic DNA. In another embodiment, at least one of the oligonucleotide probes specifically binds to the JCV genomic DNA.

In some embodiments, the kits also contain reagents to facilitate detection of amplicons or bound probes.

In another aspect, methods are provided for testing a blood sample from an organ donor for the presence of a polyomavirus using the above-described methods. In another, methods are provided for monitoring treatment of a patient with a polyomavirus comprising measuring the viral load of polyomavirus in the patient using the above-described methods. In one example, the viral load is measured before and during the treatment. Such treatments can comprise administration of an anti-viral agent, such as cidofovir, leflunomide, quinolone antibiotics and/or intravenous immunoglobulin.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIG. 1 shows the PCR amplification of BKV and JCV DNA. Samples A1 and A7 are the controls that contain no virus DNA. A2 contains BKV DNA with a final concentration of 8×10⁵ copies. A3 through A6 contain serial dilutions of BKV DNA at concentrations of 8×10⁴ copies, 8×10³ copies, 8×10² copies, 8×10¹ copies, respectively. A8 contains JCV DNA with a final concentration of 8×10⁵ copies. A9 through A12 contain serial dilutions of JCV DNA at concentrations of 8×10⁴ copies, 8×10³ copies, 8×10² copies, 8×10¹ copies, respectively

FIG. 2 shows the standard regression curve based on the amplification curves of FIG. 1. The error rate (P value) of the standard curve is 0.0949 and the efficiency is 1.935.

FIG. 3 provides a melting curve analysis. The figure shows the melting peaks of the samples that contain no virus DNA, JCV DNA only and samples contain BKV DNA only, respectively.

FIG. 4 shows the PCR amplification of BKV and JCV DNA. Samples D1 is a negative control that contains no viral DNA. D2 contains BKV DNA with a final concentration of 8×10⁵ copies. D3 through D6 contain serial dilutions of BKV DNA at concentrations of 8×10⁴ copies, 8×10³ copies, 8×10² copies, 8×10¹ copies, respectively. Wells D7 through D12 are duplicates of wells D1 through D6, respectively. Samples E1 is a blank control. Sample E2 contains BKV DNA to JCV DNA at 1:1 ratio, with a concentration of 8×10⁵ BKV DNA copies and 8×10⁵ JCV DNA copies. Samples E3-E6 contain 10-fold serial dilution of the sample in well E2, with concentrations at E3: 8×10⁴ BKV DNA copies and 8×10⁴ JCV DNA copies; E4: 8×10³ BKV DNA copies and 8×10³ JCV DNA copies; E5: 8×10² BKV DNA copies and 8×10² JCV DNA copies; E6: 8×10¹ BKV DNA copies and 8×10¹ JCV DNA copies. Wells E7-E12 are duplicates of wells E1-E6, respectively.

FIG. 5 shows the standard regression curve based on the amplification curves of FIG. 4. The error rate (P value) of the standard curve is 0.0391 and the efficiency is 1.934.

FIG. 6 provides a melting curve analysis. The figure shows the melting peaks for samples contain BKV DNA and JCV DNA at 1:1 ratio, at different concentrations. E1: Negative control sample that contain no viral DNA; E2: 8×10⁵ BKV DNA copies and 8×10⁵ JCV DNA copies; E3: 8×10⁴ BKV DNA copies and 8×10⁴ JCV DNA copies; E4: 8×10³ BKV DNA copies and 8×10³ JCV DNA copies; E5: 8×10² BKV DNA copies and 8×10²JCV DNA copies; E6: 8×10¹ BKV DNA copies and 8×10¹ JCV DNA copies. Wells E7-E12 are duplicates of wells E1-E6, respectively.

FIG. 7 demonstrates assay proficiency. In a comparative assay administrated by the College of American Pathologists (CAP), various assay were compared. The quantitative results using the method of the instant application were at or near the median value for each positive CAP sample, whereas quantitative values obtained by other laboratories using other techniques were highly variable.

FIG. 8 assay precision. The amplification curves demonstrate the precision and reproducibility of the instant method over a broad dynamic range.

### DETAILED DESCRIPTION

A stable, conserved region of the BKV genome was elucidated and determined to be an effective target for assessing whether a sample contains a polyomavirus, and in particular a BKV. Amino acid and nucleotide sequences from more than 10 species of polyomavirus were compared and evaluated for areas where the nucleotide sequence was placed under strict biological restrictions in terms of form and function, the product of the sequence experienced limited selective pressure from host immune systems, and the nucleotide sequence or product of the sequence was necessary for efficient viral replication and infection. The C-terminus of the VP2 gene (NCBI Accession No. YP_{—}717937), and in particular the region comprising amino acids 272 to 323 was identified as an ideal target. Accordingly, methods of detecting and quantifying BKV and JCV are provided, as are primers, probes and kits for use in such methods.

### Biological Sequences

A description of the biological sequences used herein is provided below.

A portion of the sequence of NCBI Accession No. NC_{—}001538, from positions 1437 to 1592, can be used as a target BKV sequence (SEQ ID NO: 1):

TCAGGAGAGTTTATAGAAAAAACTATTGCCCCAGGAGGTGCTAATCA
AAGAACTGCTCCTCAATGGATGTTGCCTTTACTTCTAGGCCTGTACG
GGACTGTAACACCTGCTCTTGAAGCATATGAAGATGGCCCCAACCAA
AAGAAAAGGAGAGTG.

In addition, other portions of the NCBI Accession No. NC_{—}001538, from positions 1437 to 1605, can be used as a target sequence.

In addition, other portions of the NCBI Accession No. NC_{—}001538, from positions 1437 to 1679, can be used as a target sequence.

In addition, other portions of the NCBI Accession No. NC_{—}001538, from positions 1 to 5153, can be used as a target sequence.

In addition, other portions of the NCBI Accession No. NC_{—}001699, from positions 1 to 5130, can be used as a target sequence.

Table 1 identifies exemplary primers and probes and provides their positions relative to NCBI Accession No. NC_{—}001538 or NC_{—}001699.

TABLE 1

| | | | | | | Position |
|---|---|---|---|---|---|---|
| | | | | | | relative |
| | | 5′ Modifi- | 3′ Modifi- | | | to NC_001538 |
| Primer/Probe | Sequence (5′→3′) | cation | cation | Type | Probe Format | or NC_001699 |
| BK_F_1.1 | CCC AGG AGG TGC TAA | None | None | Primer | N/A | 1466 to 1487 of |
| (SEQ ID NO: 2) | TCA AAG A | | | (F) | | NC_001538 |
| BK_R_1.2 | TCA TAT GCT TCA AGA GCA | None | None | Primer | N/A | 1539 to 1561 of |
| (SEQ ID NO: 3) | GGT GT | | | (R) | | NC_001538 |
| BK_P_1.3, | GCT CCT CAA TGG ATG | None | Donor | Probe | Hybridization | 1491 to 1511 of |
| Polyomavirus_F_3.1, | TTG CCT | | Fluorophore | | (Hyb Probe) | NC_001538 |
| Polyomavirus_F_4.2 | | | (e.g. FAM) | | | |
| (SEQ ID NO: 4) | | None | None | Primer | N/A | |
| | | | | (F) | | |
| BK_P_1.4 | CTT CTA GGC CTG TAC GGG | Acceptor | C3-Blocker or | Probe | Hybridization | 1515 to 1538 of |
| (SEQ ID NO: 5) | ACT GTA | Fluorophore | Phosphate | | | NC_001538 |
| BK_R_1.5, | TCA (I)AT GCT TCA AGA | None | None | Primer | N/A | 1539 to 1561 of |
| Polyomavirus_R_3.2, | GCA GGT GT | | | (R) | | NC_001538 |
| Polyoma_R_4.1 | (I) = deoxyionosine | | | | | |
| (SEQ ID NO: 6) | | | | | | |
| BK_F_1.6 | AAA AAC TAT TGC CCC | None | None | Primer | N/A | 1454 to 1476 of |
| (SEQ ID NO: 7) | AGG AGG TG | | | (F) | | NC_001538 |
| BK_F_2.1 | C CCC AGG AGG TGC TAA | None | None | Primer | SYBR Green | 1465 to 1487 of |
| (SEQ ID NO: 8) | TCA AAG A | | | (F) | | NC_001538 |
| BK_R_2.2, | TAC AGT CCC GTA CAG | None | None | Primer | SYBR Green | 1516 to 1538 of |
| BK_R_2.2.2 | GCC TAG AA | | | (R) | | NC_001538 |
| (SEQ ID NO: 9) | | Acceptor | None | Primer | FRET | |
| | | Fluorophore | | (R) | incorporated | |
| | | | | | Primer | |
| | | Phosphate | Fluorophore | Probe | Standard | |
| | | | | | Fluorescent- | |
| | | | | | Labeled | |
| BK_P_2.3 | AAG GCA ACA TCC ATT | Fluorophore | Quencher | Probe | Hydrolysis | 1488 to 1512 of |
| (SEQ ID NO: 10) | GAG GAG CAG T | | Molecule | | (Taqman) | NC_001538 |
| | | | (e.g. | | | |
| | | | TAMRA) | | | |
| BK_P_2.4 | AGG CAA CAT CCA TTG | Fluorophore | Quencher and | Probe | Hydrolysis | 1495 to 1511 of |
| (SEQ ID NO: 11) | AG | | Minor Groove | | (Taqman | NC_001538 |
| | | | Binder | | MGB) | |
| BK_F_2.5 | TCA GGA GAG TTT ATA | None | None | Primer | SYBR Green | 1437 to 1460 of |
| (SEQ ID NO: 12) | GAA AAA ACT | | | (F) | | NC_001538 |
| JCV-P-3.4 | TAC AGT CCC GTA CAA | Phosphate | Fluorophore | Probe | Standard | 1421 to 1433 of |
| (SEQ ID NO: 13) | CCC TAA AA | | | | Fluorescent- | NC_001699 |
| | | | | | Labeled | |
| BKV_P_4.3 | CCG TAC AGG CCT AGA | Fluorophore | Quencher and | Probe | Hydrolysis | 1516 to 1531 of |
| (SEQ ID NO: 14) | A | | Minor Groove | | (Taqman | NC_001538 |
| | | | Binder | | MGB) | |
| JCV_P_4.4 | CGT ACA ACC CTA AAA | Fluorophore | Quencher and | Probe | Hydrolysis | 1419 to 1435 of |
| (SEQ ID NO: 15) | GT | | Minor Groove | | (Taqman | NC_001699 |
| | | | Binder | | MGB) | |
| BKV_P_4.5 | ACA GTC CCG TAC AGG | Fluorophore | Quencher | Probe | Hydrolysis | 1515 to 1537 of |
| (SEQ ID NO: 16) | CCT AGA AG | | | | (Taqman) | NC_001538 |
| BK_R_1.7 | TTT GGC TTT TTG GGA GCT | None | None | Primer | N/A | 1601 to 1619 of |
| (SEQ ID NO: 17) | G | | | (R) | | NC_001538 |
| BK_R_1.8 | CCC TGG ACA CTC TCC TTT | None | None | Primer | N/A | 1577 to 1599 of |
| (SEQ ID NO: 18) | TCT TT | | | (R) | | NC_001538 |
| BK_F_1.1.1 | C CCC AGG AGG TGC TAA | None | None | Primer | N/A | 1465 to 1485 of |
| (SEQ ID NO: 19) | TCA AA | | | (F) | | NC_001538 |
| BK_R_1.2.1 | ATG CTT CAA GAG CAG | None | None | Primer | N/A | 1534 to 1557 |
| (SEQ ID NO: 20) | GTG TTA CAG | | | (R) | | (NC_001538) |
| BK_P_1.3.1 | CT GCT CCT CAA TGG ATG | None | Donor | Probe | Hybridization | 1489 to 1511 of |
| (SEQ ID NO: 21) | TTG CCT | | Fluorophore | | | NC_001538 |
| | | | (e.g. FAM) | | | |
| Polyoma_F_1.1.2 | CCC AGG AGG TGC (I)AA | None | None | Primer | N/A | 1466 to 1487 of |
| (SEQ ID NO: 22) | TCA AAG A | | | (R) | | NC_001538 |
| | (I) = deoxyionosine | | | | | |
| JC_P_1.5 | CTT TTA GGG TTG TAC GGG | Acceptor | C3-Blocker or | Probe | Hybridization | 1420 to 1443 of |
| (SEQ ID NO: 23) | ACT GTA | Fluorophore | Phosphate | | | NC_001699 |
| BKV_5.2 | 5′-CTG CCC CTG GAC ACT | None | None | Primer | Hybridization | 586-1603 of |
| | CTC-3′ | | | | | NC_001538 |
| BKV_5.1 | 5′-AGC TGC CCC TGG ACA | None | None | Primer | Hybridization | 1586-1605 of |
| | CTC TC-3′ | | | | | NC_001538 |

### Methods

The invention generally concerns the detection of a polyomavirus, in particular, a BKV, in a sample. In one aspect, the BKV is quantified and/or differentiated from JCV.

In one aspect, a method of testing for the presence or absence of a polyomavirus involves testing a sample for the presence or absence of a nucleic acid having the sequence of SEQ ID NO: 1 or its reverse complement. In some embodiments, the nucleic acid comprises DNA, and in other embodiments, the nucleic acid comprises RNA.

The nucleic acid of SEQ ID NO: 1 and its reverse complement can be detected using any method known in the art. In one embodiment, the nucleic acid of SEQ ID NO: 1 or its reverse complement is detected using a probe that specifically hybridizes to the nucleic acid. Typically, the detecting comprises contacting the probe with the sample under conditions in which the probe specifically hybridizes to the region, if present, and determining the presence or absence of the hybridization product. The presence of the hybridization product indicates the presence of the nucleic acid of SEQ ID NO: 1. Conversely, the absence of the hybridization product indicates the absence of the nucleic acid of SEQ ID NO: 1.

The probe is typically a nucleic acid, such as DNA, RNA, PNA or a synthetic nucleic acid. A probe specifically hybridizes to the nucleic acid of SEQ ID NO: 1 or its reverse complement if it preferentially or selectively hybridizes to the nucleic acid of SEQ ID NO: 1, or respectively its reverse complement, but does not hybridize to any other DNA or RNA sequences.

The probe preferably specifically hybridizes to the nucleic acid of SEQ ID NO: 1 under stringent hybridization conditions. Conditions that permit the hybridization are well-known in the art (for example, Sambrook et al., 2001, Molecular Cloning: a laboratory manual, 3^{rd} edition, Cold Spring Harbour Laboratory Press; and Current Protocols in Molecular Biology, Chapter 2, Ausubel et al., Eds., Greene Publishing and Wiley-Interscience, New York (1995)).

In general, “stringent hybridization conditions” denotes approximately 10° C. below the melting temperature of a perfectly base-paired double-stranded DNA hybrid (referred to as Tₘ−10). The melting temperature (Tₘ) of a perfectly base-paired double-stranded DNA can be accurately predicted using the following well-established formula:

Tₘ=16.6×log [Na³⁰]+0.41×% G:C+81.5−0.72×(%)(w/v) formamide

This formula provides a convenient means to set a reference point for determining non-stringent and stringent hybridization conditions for various DNAs in solutions having varying salt and formamide concentrations without the need for empirically measuring the Tₘ for each individual DNA in each hybridization condition.

The probe can be the same length as, shorter than or longer than the nucleic acid of SEQ ID NO: 1. The probe is typically at least 5, at least 6, at least 7, at least 8, at least 9, at least 10, at least 15, at least 20, at least 25, at least 30, at least 35, at least 45, at least 50, at least 75 or at least 100 nucleotides in length. For example, the probe can be from 5 to 200, from 7 to 100, from 10 to 50 nucleotides in length. The probe is preferably 5, 10, 15, 20, 25, 30, 35 or 40 nucleotides in length. The probe preferably includes a sequence that shares at least 80%, at least 85%, at least 90%, at least 95%, at least 98%, or at least 99% homology based on sequence identity with the nucleic acid of SEQ ID NO: 1 or its reverse complement.

Standard methods in the art may be used to determine sequence homology. For example the UWGCG Package provides the BESTFIT program which can be used to calculate homology, for example used on its default settings (Devereux et al., Nucleic Acids Research, 1984; 12: 387-395). The PILEUP and BLAST algorithms can be used to calculate homology or line up sequences (such as identifying equivalent residues or corresponding sequences (typically on their default settings)), for example as described in Altschul J Mol Evol, 1993; 36: 290-300; Altschul, et al (J Mol Biol, 1990; 215: 403-10). Software for performing BLAST analyses is publicly available through the National Center for Biotechnology Information (http://www.ncbi.nlm.nih.gov/).

The probe is detectably-labeled. The detectable label allows the presence or absence of the hybridization product formed by specific hybridization between the probe and the universal region (and thereby the presence or absence of the universal region) to be determined. Any label can be used. Suitable labels include, but are not limited to, fluorescent molecules, radioisotopes, e.g. ¹²⁵I, ³⁵S, enzymes, antibodies and linkers such as biotin.

In one aspect, the probe can be a molecular beacon probe. Molecular beacon probes comprise a fluroescent label at one end and a quenching molecule at the other. In the absence of the region to be detected, the probe forms a hairpin loop and the quenching molecule is brought into close proximity with the fluorescent label so that no signal can be detected. Upon hybridization of the probe to the region to be detected, the loop unzips and the fluorescent molecule is separated from the quencher such that a signal can be detected. Suitable fluorescent molecule and quencher combinations for use in molecular beacons are known in the art. Such combinations include, but are not limited to, carboxyfluorsecein (FAM) and dabcyl.

In another embodiment, the probe can be immobilized on a support using any technology which is known in the art. Suitable solid supports are well-known in the art and include plates, such as multi well plates, filters, membranes, beads, chips, pins, dipsticks and porous carriers.

In one embodiment, the nucleic acid itself is detected. In another embodiment, RNA transcribed from the nucleic acid is detected. The presence in the sample of RNA transcribed from the nucleic acid is itself indicative of the presence of the nucleic acid in the sample.

In some embodiments, the methods further comprise amplifying the nucleic acid of SEQ ID NO: 1 or its reverse complement or a portion of either and then testing for the presence or absence of the resulting amplicon. For example, amplification can be achieved using a pair of forward and reverse primers such as SEQ ID NO: 2 and SEQ ID NO: 3, SEQ ID NO: 2 and SEQ ID NO: 6, SEQ ID NO: 7 and SEQ ID NO: 17, or SEQ ID NO: 7 and SEQ ID NO: 18. It is to be understood that slightly longer or shorter versions of the forward and reverse primers can be used, as well. For example, the amplification step can comprise the use primers SEQ ID NO: 19 and SEQ ID NO: 20. It also is to be understood that different combinations of forward and reverse primers can be used to generate amplicons.

In one embodiment, the target is amplified before its presence is determined. In another embodiment, the target is detected in real time as its presence is determined. Real-time methods are disclosed in the Examples and have been described in the art. Such methods are described in, for example, U.S. Pat. No. 5,487,972 and Afonia et al. (Biotechniques, 2002; 32: 946-9).

The DNA or RNA can be amplified using routine methods that are known in the art. In some embodiments, the amplification of the target nucleic acid is carried out using polymerase chain reaction (PCR) (See, e.g. U.S. Pat. Nos. 4,683,195 and 4,683,202); ligase chain reaction (“LCR”) (See, e.g. Landegren et al., Science 241:1077-1080 (1988); D. Y. Wu and R. B. Wallace, Genomics 4:560-569 (1989); and F. Barany, PCR Methods Appl. 1:5-16 (1991)); loop-mediated isothermal amplification (“LAMP”) (Nagamin et al., Clin. Chem. 47(9):1742-1743 (2001); Notomi et al., Nucleic Acids Res. 28(12):E63 (2000)); nucleic acid sequence based analysis (NASBA) (J. Compton, Nature 350:91-92 (1991)); self-sustained sequence replication (“3SR”) (Guatelli et al., Proc. Natl. Acad. Sci. U.S.A. 87(5):1874-1878 (1990)); strand displacement amplification (“SDA”) (Walker et al., Nucleic Acids Res., 20:1691-1696 (1992); and Walker et al., Proc. Natl. Acad. Sci. U.S.A. 89:392-396 (1992)); or transcription mediated amplification (“TMA”) (Pasternack et al., J. Clin. Microbiol. 35(3):676-678 (1997)).

A person skilled in the art will be able to design specific primers to amplify the nucleic acid of SEQ ID NO: 1. Primers are normally designed to be complementary to sequences at either end of the sequence to be amplified but not complementary to any other sequences. Primer design is discussed in, for example, Sambrook et al., 2001, supra.

Amplicons can be detected using any method known in the art, including those described above. In some embodiments, an hydrolysis probe format (e.g., Taqman) with Minor Groove Binder (MGB) moiety can be used to detect amplicons. In other embodiments, a cyanine dye that binds to double-stranded DNA is used. Exemplary cyanine dyes include, but are not limited to, SYBR GREEN II, SYBR GOLD, YO (Oxazole Yellow), TO (Thiazole Orange), and PG (PicoGreen).

In other embodiments, the testing step can comprise conducting a melting curve analysis. Inspection of fluorescence-versus-temperature plots at the end of PCR can provide additional information when certain dyes or probe formats are used. For example, with the dye SYBR Green, the purity and identity of the PCR products can be confirmed through their melting temperatures. Similarly, when hybridization probes are used, sequence alterations, including polymorphisms, can be distinguished by probe melting temperature.

In one example, immediately after the last PCR cycle, the samples are denatured at 90° C.˜95° C., cooled to about 5° C.˜10° C. below the Tₘ range of interest and then slowly heated at a ramp rate typically ranging from 0.1 to 0.4° C./sec, while fluorescence is continuously monitored. A notable decrease in fluorescence is observed when a temperature is reached at which, depending on the particular fluorescence chemistry, either (a) a probe dissociates from the amplicon (in the case of hybridization probes) or (b) the double-stranded PCR product dissociates into single-stranded DNA.

The melting transition does not occur all at once but takes place over a small range of temperatures. The middle of the melting curve slope on the fluorescence-versus-temperature plot is referred to as the Tₘ. The melting temperature or Tm is a measure of the thermal stability of a DNA duplex and is dependent on numerous factors, including the length, G/C content and relative position of each type of nucleotide (A, T, G, C, etc.) (Wetmur, J. G. 1997. DNA Probes: applications of the principles of nucleic acid hybridization. Crit Rev Biochem Mol Biol. 26:227-259). The melting temperature is further dependent upon the number, relative position, and type of nucleotide mismatches (A:A, A:G, G:T, G:A, etc), which may occur between DNA:DNA or Probe:DNA duplexes (S. H. Ke and Wartell, R. 1993. Influence of nearest neighbor sequence on the stability of base pair mismatches in long DNA: determination by temperature-gradient gel electrophoresis. Nucleic Acids Res 21:5137-5143.) It is therefore possible to confirm the presence of a particular amplicon by melting temperature if the size and sequence of the target product is known. Likewise, it is possible to differentiate two distinct species on the basis of differential melting temperature due to sequence variation. The practicality and usefulness of melting curve analysis in PCR-based detection systems is well known.

In some embodiments, the amplification step includes the use of a pair of primers, in which at least one primer is not specific for BKV. For instance, the method comprises amplifying the nucleic acid of SEQ ID NO: 1 by contacting the sample with a pair of primers including, but not limited to, SEQ ID NO: 2 and SEQ ID NO: 3, SEQ ID NO: 2 and SEQ ID NO: 6, SEQ ID NO: 19 and SEQ ID NO: 20, SEQ ID NO: 7 and SEQ ID NO: 17, SEQ ID NO: 7 and SEQ ID NO: 18, or SEQ ID NO: 4 and SEQ ID NO: 6. In some embodiments, the methods further comprise a testing step that includes the use of at least one oligonucleotide probe capable of specifically hybridizing to BKV under stringent conditions. Exemplary probes include, but are not limited to, SEQ ID NO: 5, SEQ ID NO: 9, SEQ ID NO: 14, SEQ ID NO: 16 and SEQ ID NO: 21.

In other embodiments, the amplification step includes the use of a pair of primers, in which at least one primer is specific for BKV. For example, the methods can comprise amplifying the nucleic acid of SEQ ID NO: 1 with at least primers having the nucleic acid sequence of SEQ ID NO: 8 and SEQ ID NO: 9. In one embodiment, the testing step comprises the use of a cyanine dye that binds to double-stranded DNA.

In yet another aspect, a method is provided for testing for the presence or absence of JCV in a sample. Such methods comprise testing for the presence or absence in the sample of the nucleic acid of SEQ ID NO: 1, its reverse complement, or a sequence having 90% or more sequence homology with SEQ ID NO: 1.

In some embodiments, the amplification step includes the use of a pair of primers, in which at least one primer is not specific for BKV. In some such embodiments, the methods comprise amplifying the nucleic acid of SEQ ID NO: 1 by contacting the sample with a pair of primers including, but not limited to, SEQ ID NO: 2 and SEQ ID NO: 3, SEQ ID NO: 2 and SEQ ID NO: 6, SEQ ID NO: 19 and SEQ ID NO: 20, SEQ ID NO: 7 and SEQ ID NO: 17, SEQ ID NO: 7 and SEQ ID NO: 18, or SEQ ID NO: 4 and SEQ ID NO: 6. In some such embodiments, the methods further comprise a testing step that includes the use of at least one oligonucleotide probe capable of specifically hybridizing to JCV under stringent conditions. Exemplary probes include, but are not limited to, SEQ ID NO: 13, SEQ ID NO: 15, and SEQ ID NO: 23.

In other aspects, the methods can be employed in multiplex reactions to simultaneously test for the presence or absence of one or more species of polyomavirus. For example, the inventive methods can be used to simultaneously detect in a sample the presence or amount of each of BKV and JCV.

In some embodiments, primers are able to amplify both BKV and JCV DNA and then at least two probes, one specific for BKV and the other specific for JCV, are used to test for the presence or amount of each of BKV and JCV. For examples, different labels, such as fluorescien and rhodamine, may be used for the BKV-specific and JCV-specific probes, respectively. Alternatively, when fluorescien is used for both probes, the fluorophore for each probe must have an emission wavelength sufficiently different to distinguish between the two probes.

### Kits

Kits are provided for testing for the presence in a sample of one or more species of polyomavirus. In one embodiment, a kit comprises hybridization probes: SEQ ID NO: 5, and SEQ ID NO: 23 and a pair of primers including SEQ ID NO: 2 and SEQ ID NO: 3. In one example, SEQ ID NO: 5 and SEQ ID NO: 23 comprise acceptor fluorophore at the 5′ end and C3 blocker or phosphate at the 3′ end. In other embodiments, a kit comprises hybridization probes: SEQ ID NO: 9 and SEQ ID NO: 13 and a pair of primers including SEQ ID NO: 4 and SEQ ID NO: 6. In one example, SEQ ID NO: 9 and SEQ ID NO: 13 are labeled with two distinct fluorophores, which fluoresce at unique and distinguishable emission wavelengths. In another embodiment, a kit comprises hybridization probes: SEQ ID NO: 14 and SEQ ID NO: 15 and a pair of primers including SEQ ID NO: 4 and SEQ ID NO: 6.

The kit may additionally comprise one or more other reagents or instruments which enable the method of the invention as described above to be carried out. Such reagents or instruments include one or more of the following: suitable buffer(s) (aqueous solutions), or a support comprising wells on which reactions can be done. Reagents may be present in the kit in a dry state such that a fluid sample resuspends the reagents. The kit may, optionally, comprise instructions to enable the kit to be used in a method of the invention.

### Example 1

### Detection of Polyomaviruses

A real-time amplification assay was carried out using the primers SEQ ID NO: 2 and SEQ ID NO: 3 and probes SEQ ID NO: 4 and SEQ ID NO: 5. The assay included DNA amplification by the polymerase chain reaction (PCR) with real-time detection utilizing fluorescein-labeled donor probe SEQ ID NO: 4 and LC610-labeled acceptor probe SEQ ID NO: 5, which is designed to specifically hybridize to the BKV DNA under stringent conditions. BKV DNA and JCV DNA at various concentrations were tested, together with negative controls that contain no DNA sample.

Real-time PCR amplifications were performed on a LightCycler® 480 PCR machine (Roche, Basel, Switzerland) and data analysis was conducted using the manufacture-provided software version LCS480 1.2.9.11. Reagents from Roche (Basel, Switzerland) were used for all reactions. Each 20-μl PCR reaction contained 1× Fast-Start Hyb Probe Master Mix (Roche, Basel, Switzerland), which contains dNTPs and DNA polymerase, 0.5 μM of each of the primers (SEQ ID NO: 2 and SEQ ID NO: 3), and 0.2 μM of each of the probes (SEQ ID NO: 4 and SEQ ID NO: 5). Additional MgCl₂ was added to obtain a final concentration of 4.125 mM MgCl₂. BKV DNA and JCV DNA at different concentrations were added to each reaction well, with wells A2 through A6 containing BKV DNA at 8×10⁵ copies, 8×10⁴ copies, 8×10³ copies, 8×10² copies, and 8×10¹ copies, respectively, and wells A8 through A12 containing JCV DNA at 8×10⁵ copies, 8×10⁴ copies, 8×10³ copies, 8×10² copies, and 8×10¹ copies, respectively. Wells A1 and A7 contained no viral DNA and served as a negative control. The thermal cycler parameters comprised 1 cycle of 10 min at 95° C., 45 cycles of 10 sec at 95° C., 5 sec at 55° C. and 10 sec at 72° C. Fluorescence signals during the PCR amplification were monitored at the wavelength of 610 nm using LCS480 software in real time.

A melting curve analysis also was performed according to the manufacturer's instructions. In particular, the melting curve cycle comprised heating the samples to 95° C. for 10 sec, then cooling them to 42° C. for 1 min and then raising the temperature to 90° C. Fluorescence output for each reaction was measured continuously at 5 acquisitions per ° C. Melting temperatures for the probes were determined by LCS480 software.

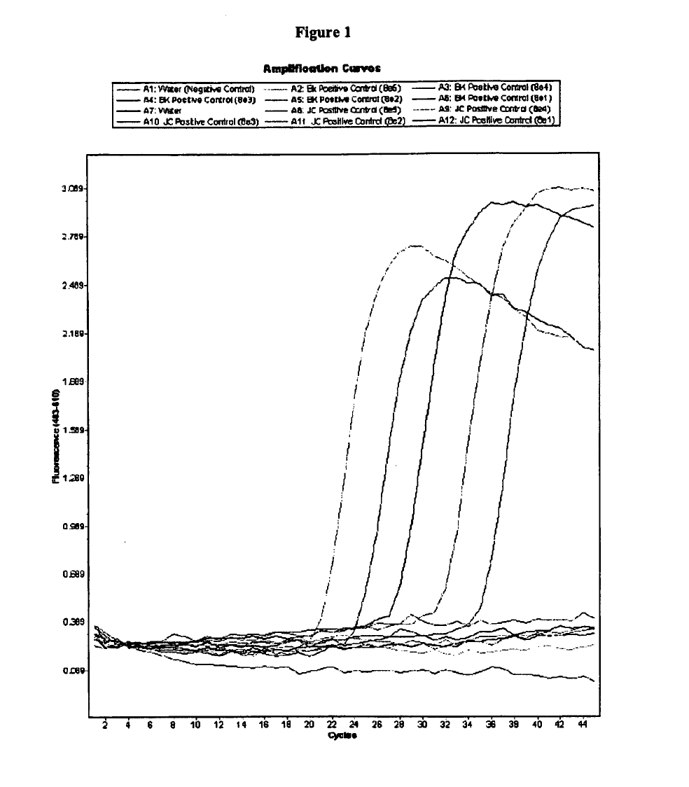

The results are shown in FIGS. 1-3.

As demonstrated in FIG. 1, the samples tested show clear and non-overlapping fluorescence signal. The samples having the earliest crossing point (Cp, which is the cycle number at which the fluorescence level rises above background) corresponds to the samples having the highest concentration of BKV DNA. Exponential rise in fluorescence was only detected in samples with BKV DNA. Fluorescence above the background level was not observed in samples that contain only JCV DNA, indicating that the probes hybridized to the BKV DNA only but not to JCV DNA at an annealing temperature of 55° C. The tests were done in duplicates, demonstrating the precision and reliability of the kit. Overall, the assay demonstrates the specificity of the probes as well as the capacity of the kit to differentiate between BKV and JCV.

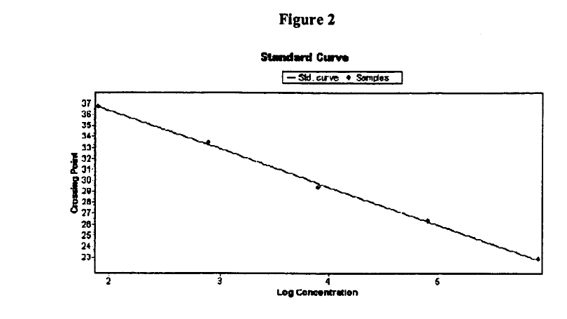

The standard curve of FIG. 2 has a low error rate (P value) of 0.0949, demonstrating the accuracy of the assay in measuring the quantity of BKV DNA across a range of concentrations from 10⁵ to 10¹ BKV DNA copies. High efficiency of the primers is proved by the empirically derived PCR amplification efficiency of 1.935.

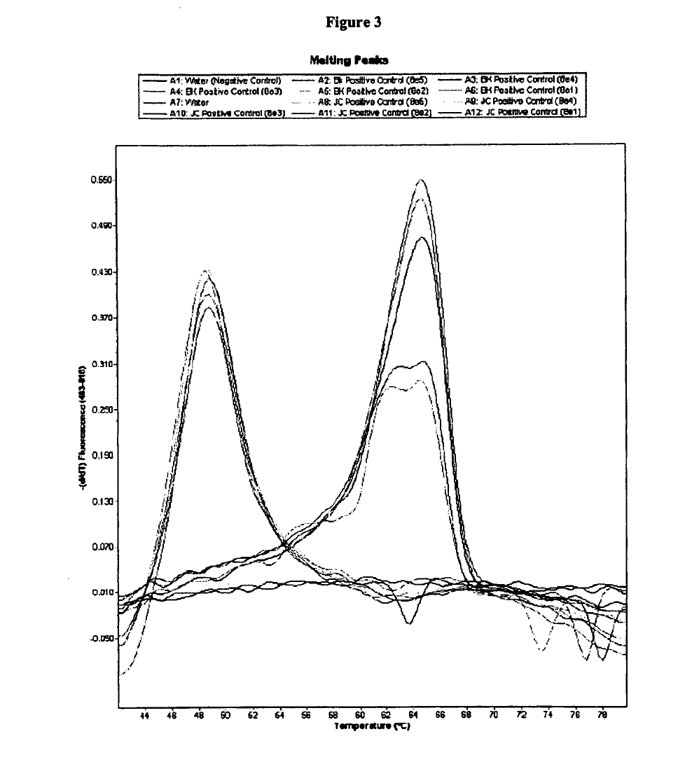

As shown in FIG. 3, positive samples can be verified as BKV by melting temperature. Fluorescence emission by the acceptor fluorophore is detected only when both SEQ ID NO: 4 and SEQ ID NO: 5 hybridize to the target amplicon allowing FRET to occur. The nucleotide sequence of probe SEQ NO: 4 is 100% homologous to BKV and JCV. Thus, SEQ NO: 4 will bind to both BKV and JCV. SEQ NO: 5 is 100% homologous to BKV DNA but has 3 nucleotide mismatches with JCV DNA. This corresponds to an observed Tm of 60° C.-64° C. for BKV and an observed Tm of 47° C.-50° C. in the case of JCV. Accordingly, there is approximate a 10° C. difference observed in the Tm of probe of SEQ ID NO: 4 between BKV DNA and JCV DNA. As a result, no increase in fluorescence is observed in samples with only JCV DNA when the primer/probe annealing step was done at or above 55° C. Therefore, SEQ NO: 4, while used as either a primer or a probe, is capable of differentiating BKV from JCV.

### Example 2

### Evaluation of Samples Comprising BKV and JCV

Samples containing both BKV and JCV were evaluated using the general conditions described in Example 1. Well D1 is a negative control that contains no viral DNA. Wells D2 through D6 contain BKV DNA at 8×10⁵ copies, 8×10⁴ copies, 8×10³ copies, 8×10² copies, and 8×10¹ copies, respectively. Wells D7 through D12 are duplicates of wells D1 through D6, respectively. Well E1 is a negative control that contains no viral DNA. Wells E2 through E6 contain both BKV DNA:JCV DNA at 1:1 ratio at concentrations of E2: 10⁵ BKV DNA copies and 10⁵ JCV DNA copies; E3:10⁴ BKV DNA copies and 10⁴ JCV DNA copies; E4:10³ BKV DNA copies and 10³ JCV DNA copies; E5:10² BKV DNA copies and 10² JCV DNA copies; E6:10¹ BKV DNA copies and 10¹ JCV DNA copies, respectively. Wells E7 through E12 are duplicates of wells E1 through E6, respectively.

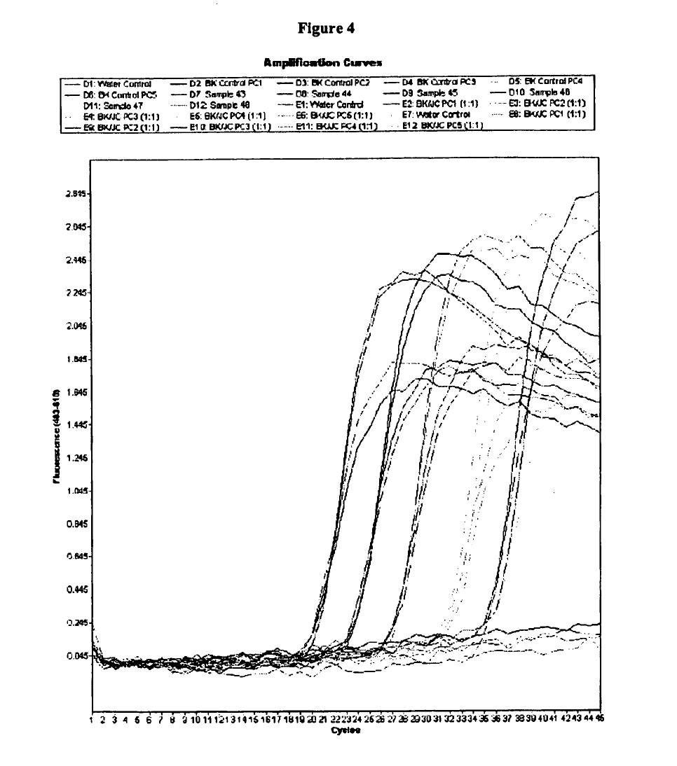

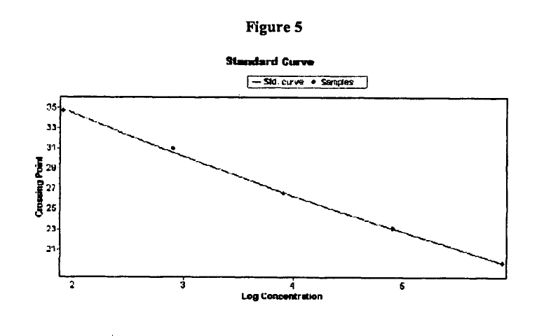

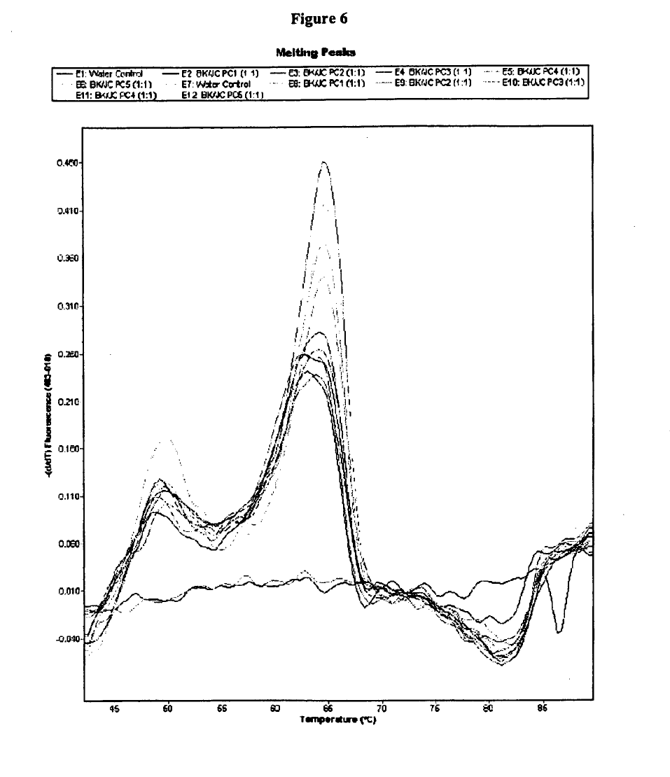

The amplification curve, standard regression curve, and the melting peaks are shown in FIG. 4, FIG. 5, and FIG. 6, respectively.

FIG. 4 demonstrates that the probes hybridized to BKV but not to JCV DNA at an annealing temperature of 55° C. or higher. Comparing to the amplification curves of FIG. 1, the presence of JCV DNA in 1:1 ratio with BKV DNA in samples does not impact the reproducibility or precision of the kit. Therefore, the high level of accuracy and precision that is maintained in a sample containing both BKV and JCV DNA, at a ratio up to a 1:1.

FIG. 5 shows that the high level of accuracy observed in FIG. 2 is reproducible.

The graph of FIG. 6 illustrates the characteristic double melting peak observed in samples containing a mix of both BKV and JCV DNA. The double melting peaks, one peak at the expected Tm for BKV DNA and the second at the expected Tm for JCV DNA, is indicative of a mixed sample containing both BKV and JCV.

### Example 3

### Clinical Testing

Routine testing of clinical samples was conducted using the following primer/probe combinations: combination 1 consisting of primers SEQ ID NO: 6 and SEQ ID NO: 4, as well as, probe sequence SEQ ID NO: 14; combination 2 consisting of primers SEQ ID NO: 6 and SEQ ID NO: 2, as well as, probe sequence SEQ ID NO: 14; combination 3 consisting of primers BKV_{—}5.2 and SEQ ID NO: 4, as well as, probe sequence SEQ ID NO: 14; and, combination 4 consisting of primers BKV_{—}5.2 and SEQ ID NO: 2, as well as, probe sequence SEQ ID NO: 14.

The PCR reaction comprises a final reaction volume of 40 μl; with 10 μl of sample & 30 μl master mix. The master mix composition (30 μl) comprises a forward primer at a concentration of 3.125 μM, a reverse primer at a concentration of 3.125 μM, a MGB Taqman probe at a concentration of 2.0˜2.5 μM, 20 μl of LightCycler®480 Probes master mix and 10 μl of sample DNA for a total volume of 40 μl per sample well. The PCR cycling parameters for primer probe combinations was i) an initial single denaturing cycle of 95° C. for 10 minutes followed by ii) 45 cycles of: 95° C. for 10 seconds, 60° C. for 15 seconds and 72° C. for 1 second with a single fluorescence measurement being taken at the end of each cycle, and optionally, iii) a final cool down of the 96-well plate at 40° C. for 30 seconds.

A group of 82 clinical specimens, 42 urine and 40 plasma specimens, were tested to detect polyomavirus using the aforementioned protocol. Of the urine samples tested, 35 were identified as positive and 7 were identified as negative. Of the plasma samples, 22 were identified as positive and 18 were identified as negative. In total, 57 of the 82 clinical samples tested were identified as polyomavirus positive. All samples identified as positive were determined to be from clinically confirmed cases of viuria &/or viremia.

### Example 4

### College of American Pathologists (CAP) Survey for BKV Viral Load

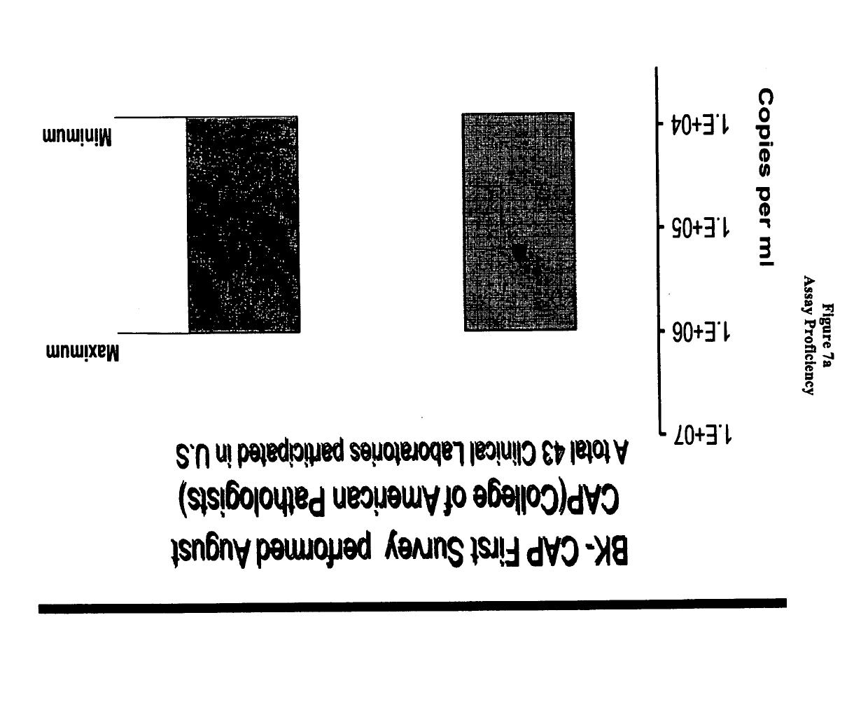

Two samples were assayed in two separate cap surveys to test for BKV viral load. Using the aforementioned assay protocol detailed in example 3, all BKV positive samples were identified in concordance with all 43 survey participants using a diverse array of techniques including for example, commercially available kits for detection of BKV. As exemplified in FIG. 7, the results of the CAP proficiency test, demonstrate the method of the instant application was at or near the median value for each positive CAP sample. Importantly, while the method of the instant application was at or near the median value for each sample, the quantitative values obtained by other participants using different methods were highly variable.

### Example 5

### Comparative Study with External Laboratory

The method of the instant application was further validated by a comparison study with an external, independent laboratory. A total of 74 clinical samples were tested. The clinical status of each sample, such as polyomavirus positive or polyomavirus negative, was unknown at the time of testing. In total the 74 unknown samples comprised a sample set of 30 urine samples and 44 plasma samples. Of the 30 urine samples, 10 were positive for BKV and 20 were negative. Of the 44 plasma samples, 24 were positive for BKV and 20 were negative. Using the method of the instant application, a sensitivity of 100% was achieved. The sensitivity and specificity was calculated using the following formula:

Urine sample sensitivity (%)=(True Positive/(True Positive+False Negative))×100=(34/(34+0))×100=100%

Plasma sample specificity (%)=(True Negative/(True Negative+False Positive))×100=(40/(40+0)×100=100%

The precision of the instant method was measured using commercial standard of known concentration to determine assay precision. Serial dilutions of known BK virus DNA was amplified according the aforementioned method using SEQ ID NO: 4, BKV_{—}5.2 and SEQ ID NO: 14 primer probe set. The amplification was performed in triplicate, and Table 2 summarizes the precision of the instant method. The method of the instant application demonstrates that experiments performed multiple times vary only slightly and their results may be directly compared.

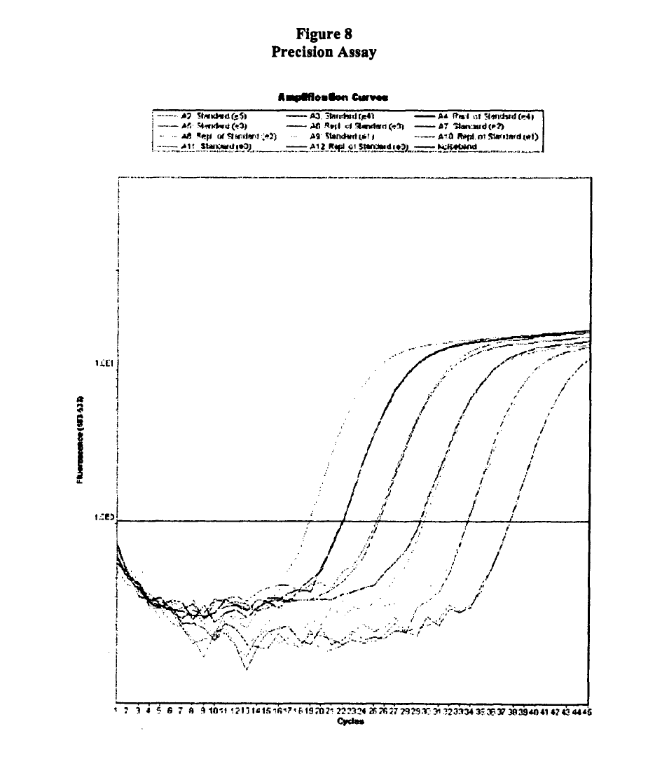

FIG. 8 discloses a second example illustrating the precision and reproducibility of the instant method. The amplification curves demonstrate the precision and reproducibility of the instant method over a broad dynamic range.

Thus the invention is directed to a method of testing for the presence or absence of a polyomavirus DNA in a sample, wherein the results of said test can be reproduced with greater than 95% precision, preferably greater than 97% precision, at a predetermined crossing point (Cp). More preferably, the method of testing determines whether the starting quantity of DNA measured is low, medium or high.

TABLE 2
Assay Precision

| Replicate number | Relative cocentration | Very High | High | Mid | Mid | Low | Very Low |
|---|---|---|---|---|---|---|---|
| | Expected Concentration | 7.5E+05 | 7.5E+04 | 7.5E+03 | 7.5E+02 | 7.5E+01 | 7.5 |
| | (copies/μl/well) | | | | | | |
| | Experiment 1 | | | | | | |
| Replicate 1 | Observed Concentration | 7.60E+05 | 8.10E+04 | 7.27E+03 | 7.65E+02 | 5.95E+01 | 6.59E+00 |
| | Cp (PCR amplification cycles) | 22.71 | 25.96 | 29.46 | 32.73 | 35.44 | 37.06 |
| Replicate 2 | Observed Concentration | 7.07E+05 | 7.75E+04 | 6.69E+03 | 7.51E+02 | 5.17E+01 | 8.96E+00 |
| | Cp (PCR amplification cycles) | 22.82 | 26.03 | 29.53 | 32.76 | 35.56 | 36.86 |
| Replicate 3 | Observed Concentration | — | 8.25E+04 | 7.59E+03 | 7.11E+02 | 8.75E+01 | 5.44E+00 |
| | Cp (PCR amplification cycles) | — | 25.93 | 29.4 | 32.84 | 35.11 | 37.19 |
| Replicate 4 | Observed Concentration | — | 7.53E+04 | 7.11E+03 | 8.67E+02 | 8.20E+01 | 1.54E+01 |
| | Cp (PCR amplification cycles) | — | 26.07 | 29.49 | 32.55 | 35.17 | 36.48 |
| | Experiment 2 | | | | | | |
| Replicate 1 | Observed Concentration | 7.16E+05 | 7.71E+04 | 6.67E+03 | 7.57E+02 | 7.31E+01 | 9.35E+00 |
| | Cp (PCR amplification cycles) | 21.61 | 24.89 | 28.49 | 31.69 | 35.13 | 38.16 |
| Replicate 2 | Observed Concentration | — | 8.26E+04 | 8.17E+03 | 7.86E+02 | 8.33E+01 | 1.33E+01 |
| | Cp (PCR amplification cycles) | — | 24.78 | 28.19 | 31.64 | 34.94 | 37.64 |
| Replicate 3 | Observed Concentration | — | — | — | 7.88E+02 | 7.28E+01 | 4.71E+00 |
| | Cp (PCR amplification cycles) | — | — | — | 31.63 | 35.14 | 39.17 |
| Replicate 4 | Observed Concentration | — | — | — | 7.48E+02 | 6.36E+01 | 8.52E+00 |
| | Cp (PCR amplification cycles) | — | — | — | 31.71 | 35.34 | 38.29 |
| | Experiment 3 | | | | | | |
| Replicate 1 | Observed Concentration | 7.89E+05 | 7.15E+04 | 7.21E+03 | 7.46E+02 | 5.22E+01 | 1.18E+01 |
| | Cp (PCR amplification cycles) | 22.34 | 25.88 | 29.25 | 32.59 | 35.69 | 37.04 |
| Replicate 2 | Observed Concentration | 7.62E+05 | 7.47E+04 | 7.08E+03 | 8.20E+02 | 8.46E+01 | 5.12E+00 |
| | Cp (PCR amplification cycles) | 22.39 | 25.81 | 29.28 | 32.46 | 35.21 | 37.73 |

Precision Summary:

| | | | | | | |
|---|---|---|---|---|---|---|
| Average (Cp) | 22.37 | 25.67 | 29.14 | 32.26 | 35.27 | 37.56 |
| Std Dev (Cp) | 0.47 | 0.52 | 0.59 | 0.52 | 0.23 | 0.89 |
| CV (ave/SD) | 2.1% | 2.0% | 2.0% | 1.6% | 0.7% | 2.4% |

1. A method of testing for the presence or absence of a polyomavirus in a sample, comprising testing the sample for the presence or absence of a nucleic acid having the sequence of SEQ ID NO: 1, its reverse complement, or a sequence having 90% or more sequence homology with SEQ ID NO: 1.

2. The method of claim 1, further comprising amplifying the nucleic acid of SEQ ID NO: 1 or its reverse complement or a portion of either and then testing for the presence or absence of the resulting amplicon.

3. The method of claim 2, in which the testing step includes contacting the sample with at least one oligonucleotide probe capable of hybridizing to the nucleic acid of SEQ ID NO: 1 or its reverse complement under stringent conditions.

4. The method of claim 3, wherein the testing step further comprises conducting a melting curve analysis.

5. The method of claim 3, wherein said amplification step comprises the use of at least amplification primers SEQ ID NO: 2 (BK_F_{—}1.1) and SEQ ID NO: 3 (BK_R_{—}1.2) and the testing step comprises the use of at least oligonucleotide probes SEQ ID NO: 4 (BK_P_{—}1.3) and SEQ ID NO: 5 (BK_P_{—}1.4).

6. The method of claim 3, wherein said amplification step comprises the use of at least amplification primers SEQ ID NO: 2 (BK_F_{—}1.1) and SEQ ID NO: 6 (BK_R_{—}1.5) and the testing step comprises the use of at least oligonucleotide probes SEQ ID NO: 4 (BK_P_{—}1.3) and SEQ ID NO: 5 (BK_P_{—}1.4).

7. The method of claim 3, wherein said amplification step comprises the use of at least amplification primers SEQ ID NO: 2 (BK_F_{—}1.1) and SEQ ID NO: 3 (BK_R_{—}1.2) and the testing step comprises the use of at least oligonucleotide probes SEQ ID NO: 4 (BK_F_{—}1.3) and SEQ ID NO: 23 (JC_P_{—}1.5).

8. The method of claim 3, wherein said amplification step comprises the use of at least amplification primers SEQ ID NO: 2 (BK_F_{—}1.1) and SEQ ID NO: 3 (BK_R_{—}1.2) and the testing step comprises the use of at least oligonucleotide probes SEQ ID NO: 5 (BK_P_{—}1.4) and SEQ ID NO: 23 (JC_P_{—}1.5).

9. The method of claim 1, wherein said amplification step comprises the use of at least amplification primers SEQ ID NO: 8 (BK_F_{—}2.1) and SEQ ID NO: 9 (BK_R_{—}2.2).

10. The method of claim 9, wherein the testing step comprises the use of a cyanine dye that binds to double-stranded DNA.

11. The method of claim 3, wherein said amplification step comprises the use of at least amplification primers SEQ ID NO: 4 (Polyomavirus_F_{—}3.1) and SEQ ID NO: 6 (Polyomavirus_R_{—}3.2) and the testing step comprises the use of at least oligonucleotide probes SEQ ID NO: 9 (BK_P_{—}3.3) and SEQ ID NO: 13 (JCV_P_{—}3.4).

12. The method of claim 3, wherein said amplification step comprises the use of at least amplification primers SEQ ID NO: 4 (Polyomavirus_F_{—}3.1) and SEQ ID NO: 6 (Polyomavirus_R_{—}3.2) and the testing step comprises the use of at least oligonucleotide probe SEQ ID NO: 9 (BK_P_{—}3.3).

13. The method of claim 3, wherein said amplification step comprises the use of at least amplification primers SEQ ID NO: 4 (Polyomavirus_F_{—}4.1) and SEQ ID NO: 6 (Polyomavirus_R_{—}4.2) and the testing step comprises the use of at least oligonucleotide probes SEQ ID NO: 14 (BK_P_{—}4.3) and SEQ ID NO: 15 (JCV_P 4.4).

14. A kit comprising at least one oligonucleotide probe capable of hybridizing to the nucleic acid of SEQ ID NO: 1 under stringent conditions.

15. The kit of claim 14, further comprising amplification primers for amplifying the nucleic acid of SEQ ID NO: 1, a complement or transcript or a portion thereof.

16. The kit of claim 15 comprising amplification primers SEQ ID NO: 2 (BK_F_{—}1.1) and SEQ ID NO: 3 (BK_R_{—}1.2) and oligonucleotide probes SEQ ID NO: 4 (BK_P_{—}1.3) and SEQ ID NO: 5 (BK_P_{—}1.4).

17. The kit of claim 15 comprising amplification primers SEQ ID NO: 2 (BK_F_{—}1.1) and SEQ ID NO: 6 (BK_R_{—}1.5) and oligonucleotide probes SEQ ID NO: 4 (BK_P_{—}1.3) and SEQ ID NO: 5 (BK_P_{—}1.4).

18. The kit of claim 15 comprising amplification primers SEQ ID NO: 2 (BK_F_{—}1.1) and SEQ ID NO: 3 (BK_R_{—}1.2) and oligonucleotide probes SEQ ID NO: 4 (BK_P_{—}1.3) and SEQ ID NO: 23 (JC_P_{—}1.5).

19. The kit of claim 15 comprising amplification primers SEQ ID NO: 2 (BK_F_{—}1.1) and SEQ ID NO: 3 (BK_R_{—}1.2) and oligonucleotide probes SEQ ID NO: 5 (BK_P_{—}1.4) and SEQ ID NO: 23 (JC_P_{—}1.5).

20. The kit of claim 15 comprising amplification primers SEQ ID NO: 4 (Polyomavirus_F_{—}3.1) and SEQ ID NO: 6 (Polyomavirus_R_{—}3.2) and oligonucleotide probes SEQ ID NO: 9 (BK_P_{—}3.3) and SEQ ID NO: 13 (JCV_P_{—}3.4).

21. The kit of claim 15 comprising amplification primers SEQ ID NO: 4 (Polyomavirus_F_{—}3.1) and SEQ ID NO: 6 (Polyomavirus_R_{—}3.2) and oligonucleotide probe SEQ ID NO: 9 (BK_P_{—}3.3).

22. The kit of claim 15 comprising amplification primers SEQ ID NO: 4 (Polyomavirus_F_{—}4.1) and SEQ ID NO: 6 (Polyomavirus_R_{—}4.2) and oligonucleotide probes SEQ ID NO: 14 (BK_P_{—}4.3) and SEQ ID NO: 15 (JCV_P_{—}4.4).

23. A kit comprising amplification primers SEQ ID NO: 8 (BK_F_{—}2.1) and SEQ ID NO: 9 (BK_R_{—}2.2).

24. A method of testing a blood sample from an organ donor for the presence of a polyomavirus comprising the method of any of claims 1.

25. The method of claim 24, wherein the organ considered for donation is selected from the group consisting of kidney, liver, and heart.

26. The method of claim 24, further comprising rejecting an organ from an organ donor found positive for a polyomavirus.

27. A method of monitoring treatment of a patient with a polyomavirus comprising measuring the viral load of polyomavirus in said patient using a method of any of claims 1.

28. The method of claim 27, wherein the viral load is measured before and during said treatment.

29. The method of claim 27, wherein said treatment comprises administration of an anti-viral agent.

30. The method of claim 29, wherein said anti-viral agent is selected from the group consisting of cidofovir, leflunomide, quinolone antibiotics and intravenous immunoglobulin.

31. The method of claim 1, wherein said amplification step comprises the use of at least two amplification primers selected from the group consisting of SEQ ID NO: 2, SEQ ID NO: 4, SEQ ID NO: 6 and primer BKV_{—}5.2 and probe SEQ ID NO: 14.

32-34. (canceled)
